(19) United States
(12) Patent Application Publication
(10) Pub. No.: US 2025/0093285 A1
(43) Pub. Date: Mar. 20, 2025

(54) SUBCELLULAR SELF-TRACER ION IMAGING AND LOCALIZATION METHOD OF METAL ELEMENTS

(71) Applicant: Shandong Laboratory of Advanced Agricultural Sciences in Weifang, Weifang (CN)

(72) Inventors: Xiaohua HUANG, Weifang (CN); Xing Wang DENG, Hamden, CT (US); Mengzhu CHENG, Weifang (CN); Jun ZHAO, Weifang (CN); Bin XU, Weifang (CN)

(73) Assignee: Shandong Laboratory of Advanced Agricultural Sciences in Weifang, Weifang (CN)

(21) Appl. No.: 18/966,139

(22) Filed: Dec. 3, 2024

(30) Foreign Application Priority Data
Dec. 6, 2023 (CN) 202311670404.7

(51) Int. Cl. G01N 23/2251; G01N 1/30; G01N 1/36; G01N 23/2202; G01N 23/2258

(52) U.S. Cl. CPC G01N 23/2251; G01N 1/30; G01N 1/36; G01N 23/2202; G01N 23/2258; G01N 2001/305

(57) ABSTRACT

Provided is a subcellular self-tracer ion imaging and localization method of metal elements. Wherein, the above method includes: using the SEM-FIB-TOF-SIMS system to perform subcellular structure imaging and metal ion imaging on the sample slice, wherein in the SEM-FIB-TOF-SIMS system, the scanning electron microscope (SEM) is used to perform subcellular structure imaging on the sample slice; and the focused ion beam (FIB) is used to perform surface bombardment on the subcellular structures, and the secondary ions excited are detected by the time-of-flight secondary ion mass spectrometry (TOF-SIMS) to obtain the ion information in the analysis area and imaged.

### CROSS-REFERENCE TO RELATED APPLICATION(S)

The present application claims priority to and interest of patent application Ser. No. 20/231,1670404.7 filed to the China National Intellectual Property Administration on Dec. 6, 2023, the disclosure of which is hereby incorporated by reference in its entirety.

### FIELD

The present application relates to the technical field of ion imaging, specifically to a subcellular self-tracer ion imaging and localization method of metal elements.

### BACKGROUND

At present, research on subcellular localization of metal elements mainly includes in vivo and in situ visualization and in vitro quantification research after extracting different components of cells. However, in the in vivo and in situ visualization research, there are the following issues: (1) the indirect tracer methods using probe labeling, where most elements such as transition elements, etc., have no commercially available probes; (2) the imaging methods of radioactive isotope self-tracer imaging, which are complex and harmful to the human body; and (3) the imaging methods of non-radioactive isotope self-tracer imaging, which are difficult to accurately determine the subcellular localization due to poor repeatability and unclear images.

### SUMMARY

The main object of the present application is to provide a subcellular self-tracer ion imaging and localization method for metal elements, in order to solve the problem that the subcellular localization methods of metal elements in the prior art are difficult to meet the needs of practical applications.

In order to achieve the above object, according to a first aspect of the present application, a subcellular self-tracer ion imaging and localization method of metal elements is provided. The aforementioned method includes: using an SEM-FIB-TOF-SIMS system to perform a subcellular structure imaging and a metal ion imaging on a sample slice, wherein in the SEM-FIB-TOF-SIMS system, the scanning electron microscope (SEM) is used to perform subcellular structure imaging on the sample slice; and the focused ion beam (FIB) is used to perform surface bombardment on the subcellular structure, and the secondary ions excited are detected by the time-of-flight secondary ion mass spectrometry (TOF-SIMS) to obtain the ion information in the analysis area and imaged.

Further, the sample slice includes a plant slice, an animal tissue slice, a bacterial slice, or a fungal sample, and correspondingly, the subcellular structure includes a plant subcellular structure, an animal subcellular structure, a bacterial subcellular structure, or a fungal subcellular structure.

Further, the metal ion includes a transition metal element ion or a non-transition metal element ion. Further, the transition metal element ion includes a rare earth element ion.

Further, the method for preparing the plant slice includes: cutting a plant tissue into a small piece and subjecting the same to fixation treatment, thereby obtaining a fixed sample; and subjecting the fixed sample to dehydration treatment, infiltration and embedding treatment, and slicing treatment successively, thereby gradually obtaining a dehydrated sample, an infiltrated and embedded sample, and the plant slice.

Further, the fixation treatment includes pre-fixation treatment and post-fixation treatment; the pre-fixation treatment includes: pre-fixing the small piece using polyformaldehyde and glutaraldehyde to obtain a pre-fixed sample; preferably, the post-fixation treatment includes: post-fixing the pre-fixed sample using an osmic acid solution to obtain a post-fixed sample; preferably, the dehydration treatment includes: dehydrating the fixed sample using ethanol solutions with concentrations that gradually increasing sequentially to obtain a dehydrated sample; preferably, the infiltration and embedding treatment includes: using propylene oxide and an embedding agent for the infiltration and embedding of the dehydrated sample, resulting in an infiltrated and embedded sample; preferably, and the slicing treatment includes: slicing the infiltrated and embedded sample to obtain the plant slice.

Further, a size of the small piece is 1-2 mm³; preferably, the pre-fixation treatment includes: placing the small piece into a solution containing 1-4 wt % of paraformaldehyde and 0.5-5 wt % of glutaraldehyde, fixing same in a dark place at 15-25° C. for 2-3 hours or overnight at 4° C. to obtain the pre-fixed sample; preferably, the post-fixation treatment includes: placing the pre-fixed sample in a 1-2 wt % osmic acid solution, fixing same in a dark place at 15-25° C. for 4 hours to obtain the post-fixed sample; preferably, the dehydration treatment includes: subjecting the post-fixed sample to the dehydration treatment using the ethanol solutions of 30% (v/v), 50% (v/v), 70% (v/v), 90% (v/v), 100% (v/v), and 100% (v/v), sequentially, wherein treatment times for the ethanol solutions ranging from 30% (v/v) to 90% (v/v) are 15 minutes each, and treatment times for the 100% (v/v) ethanol solution are 20 minutes each, thereby obtaining the dehydrated sample; and preferably, the infiltration and embedding treatment includes: sequentially treating the dehydrated sample with propylene oxide at 15-25° C. for 1 hour, with gradient mixed solutions of propylene oxide and an embedding agent at 15-25° C. for 4 hours, respectively, with an embedding agent at 15-25° C. for 8 hours, then polymerizing same at 60° C. for 48 hours to obtain the infiltrated and embedded sample; and a thickness of the plant slice is 80-150 nm.

Further, before preparing the plant slices, the plant is treated with the metal ion, including: spraying a solution containing the metal ion onto a leaf or leaves of the plant until droplets begin to fall; and preferably, a concentration of the metal ion in the solution is 1 nmol/L to 1 mmol/L.

Further, when the SEM is used for the subcellular structure imaging, an electron beam current is 21 pA to 1.4 nA, an acceleration voltage is 3-10 kV, a scanning speed is 1-100 μs, and a magnification factor is 2,500 to 5,000 folds.

Further, when the FIB-TOF-SIMS is used for the surface bombardment, the secondary ion signal detection, and the ion imaging of the subcellular structure, an ion beam current is 24-80 pA and an acceleration voltage is 30 kV, and a number of images collected is 50-200.

Further, after the subcellular structure imaging and the ion imaging are performed, an image area and a data sheet of the image area are output, and a mass spectrometry image in the image area that can characterize the relative abundance of the metal ions is drawn based on the data sheet.

By applying the technical solution of the present application, the SEM-FIB-TOF-SIMS system is used to achieve the subcellular self-tracer ion imaging and localization. Firstly, the SEM is used to perform subcellular structure imaging on the sample slice; then the FIB is used to perform surface bombardment on the subcellular structure, and the secondary ions excited are detected by the TOF-SIMS to obtain the ion information in the analysis area and imaged, which can achieve the ion imaging and localization at the subcellular structure level, the image is clear, the localization is accurate, the operation is simple, and there is no need for using radioactive isotopes.

### BRIEF DESCRIPTION OF DRAWINGS

The accompanying drawings of the description, which form a part of the application, are used to provide a further understanding of the present application. The illustrative embodiments and their descriptions of the application are used to explain the application, and do not constitute an improper limitation thereto. In the accompanying drawings:

FIG. 1 shows the graph of the subcellular self-tracer ion imaging results of the rare earth elements according to Example 1 of the present application, where PM represents the plasma membrane, V represents the vacuole, CP represents the chloroplast, and Ce represents the cerium ion.

FIG. 2 shows the mass spectrum of the relative abundance of the rare earth elements in chloroplasts in Example 1 of the present application.

FIG. 3 shows the graph of the rare earth radioactive isotope self-tracer imaging results according to Example 1 of the present application, where PM represents the plasma membrane, V represents the vacuole, T represents the vacuole membrane, ChM represents the chloroplast membrane, and the small arrows with no abbreviations represent the rare earth elements.

FIG. 4 shows the graph of the rare earth radioactive isotope self-tracer imaging results according to Example 1 of the present application, where T represents the vacuole membrane, ChM represents the chloroplast membrane, Chl represents the chloroplast, and the small arrows with no abbreviations represent the rare earth elements.

FIG. 5 shows the graph of the rare earth non-radioactive isotope self-tracer imaging results according to Example 1 of the present application, where CW represents the cell wall and PM represents the plasma membrane.

FIG. 6 shows the graph of the subcellular self-tracer ion imaging results of the transition metal element (iron, Fe) according to Example 1 of the present application.

FIG. 7 shows the graph of the subcellular self-tracer ion imaging results of the transition metal element (zinc, Zn) according to Example 1 of the present application.

FIG. 8 shows the graph of the subcellular self-tracer ion imaging results of the non-transition metal element (calcium, Ca) according to Example 1 of the present application.

FIG. 9 shows the graph of the subcellular self-tracer ion imaging results of the non-transition metal element (magnesium, Mg) according to Example 1 of the present application.

FIG. 10 shows the graph of the subcellular self-tracer ion imaging and localization results of the rare earth elements in a corn according to Example 2 of the present application, where Ce represents the cerium ion.

FIG. 11 shows the graph of the subcellular self-tracer ion imaging and localization results of the rare earth elements according to Example 3 of the present application, where Ce represents the cerium ion.

FIG. 12 shows the graph of the subcellular self-tracer ion imaging and localization results of the rare earth elements according to Example 4 of the present application, where Ce represents the cerium ion.

FIG. 13 shows the graph of the subcellular self-tracer ion imaging and localization results of the rare earth elements according to Example 5 of the present application, where Ce represents the cerium ion.

### DESCRIPTION OF EMBODIMENTS

It should be noted that the examples and features in the examples in the present application can be combined with each other without conflicting. The present application will be described in detail below in conjunction with examples.

Terminology explanation:

SEM refers to scanning electron microscope.

FIB refers to focused-ion-beam.

TOF-SIMS refers to time of flight-secondary ion mass spectrometry.

Subcell refers to a structure that is more refined than a cellular structure.

Subcellular structure refers to a cellular structure that is only visible under electron microscopy, such as mitochondria, centrosomes, golgiosomes, and pits on cell walls, etc.

Subcellular localization refers to the specific location of proteins, gene expression products, or compounds present within a cell, such as present in the nucleus, cytoplasm, cell membrane, or other specific subcellular structures.

As mentioned in the background, the researches on subcellular visualization localization of metal elements in the prior art are affected by issues such as limited types of probes, high cost and significant harm of radioactive isotope reagents, and unclear imaging, etc., making it difficult to accurately locate metal elements at the subcellular level. Therefore, in this application, the inventors have attempted to develop a novel subcellular self-tracer ion imaging and localization method of metal elements, and proposed the solution claimed in this application.

In a typical embodiment of this application, a subcellular self-tracer ion imaging and localization method of metal elements is provided, the aforementioned method includes: using an SEM-FIB-TOF-SIMS system to perform a subcellular structure imaging and a metal ion imaging on a sample slice; wherein in the SEM-FIB-TOF-SIMS system, the scanning electron microscope (SEM) is used to perform subcellular structure imaging on the sample slice; and the focused ion beam (FIB) is used to perform surface bombardment on the subcellular structure, and the secondary ions excited are detected by a time-of-flight secondary ion mass spectrometry (TOF-SIMS) to obtain an ion information in an analysis area and imaged.

In the SEM-FIB-TOF-SIMS system as used in this application, the model of the SEM-FIB is Helios5CX (Thermo Fisher Scientific, the United States of America), and the model of the TOF-SIMS is C-TOF (Tofwerk, Switzerland). By connecting the aforementioned devices, the above system can thus be obtained. The aforementioned method can also be achieved using other devices with the same function. The aforementioned subcellular self-tracer ion imaging and localization method of metal elements is not limited to the detection using specific models of devices. In the aforementioned method, the subcellular structure is first determined by SEM, and then specific subcellular structures are bombarded to achieve imaging and localization of metal ions in the subcellular structure. With the aforementioned methods, it is possible to simultaneously image and locate one or more kinds of metal ions in the subcellular structure.

In a preferred embodiment, the sample or the sample slice includes but is not limited to a plant slice, an animal tissue slice, a bacterial slice or a fungal sample, and correspondingly, the subcellular structure includes a plant subcellular structure, an animal subcellular structure, a bacterial subcellular structure, or a fungal subcellular structure. Preferably, the plant can be a model plant or a non-model plant; or it can be a non-crop plant or a crop plant. The sample can be originated from leaves, root systems, or other parts, and specifically including but not limited to Arabidopsis thaliana or Zea mays, and the method of this application is applicable to all cellular structures.

In a preferred embodiment, the metal ion includes a transition metal element ion or a non-transition metal element ion;

The transition metal elements, i.e. a general designation for 68 metal elements with valence electron arrangements located in the d, ds, or f regions of the periodic table of elements, include but are not limited to rare earth elements. The non-transition metal elements, i.e. metal elements that do not belong to the transition metal elements, include but are not limited to the metal elements in the main groups I, II, III, IV, V, and VI. Throughout the entire life cycle of plants, except for artificial elements, they are continuously absorbed by plant cells and transported to different subcellular regions, where they accumulate and affect the growth and development of plants. For a long time, revealing the absorption, transport, and accumulation mechanisms of metal elements (especially transition metal elements) by plant cells has always been a hot topic and difficult problem for researchers from multiple disciplines such as biology, chemistry, environment, and agriculture, etc., worldwide. Among them, revealing the subcellular localization of metal elements in plants is widely recognized as one of the important means to answer the aforementioned hot topic and difficult problem.

In the aforementioned subcellular self-tracer ion imaging and localization method of metal elements, the Scanning Electron Microscope-Focused Ion Beam-Time of Flight-Secondary Ion Mass Spectrometry (SEM-FIB-TOF-SIMS) system is used, wherein firstly, the SEM is used to perform subcellular structure imaging on a single cell in the sample slice; further, the FIB is used to bombard the obtained subcellular structure with an ion beam, and the secondary ions excited after the bombardment are collected using the TOF-SIMS to obtain the ion information in the bombardment area and imaged in the corresponding analysis software, thereby achieving the imaging and localization of metal ions in the subcell. This method has the advantages of simple and fast operation, clear image and good repeatability. It lays a technical foundation for studying the cellular and molecular mechanisms for transition metal elements, etc., in regulating plant growth.

The above mentioned metal ions are compounds (encompassing substance species defined in expression of specialized English, such as Compound), complexes (encompassing substance species defined in expressions of specialized English, such as Coordination compound or Coordination complex), or complexes (encompassing substance species defined in the expression of specialized English, such as Complex) of metal ions with stable valence.

In a preferred embodiment, the transition metal element ion includes a rare earth element (REE) ion. The working principle of FIB-TOF-SIMS is to irradiate the surface of the sample with a high-energy focused ion beam, causing the atomic layer on the surface of the sample to be tested to excite secondary ions. Next, the corresponding ions are detected and imaged based on the time it takes for different masses of secondary ions to fly to the detector. Among them, both the ion sputtering of FIB and the ion detection of TOF-SIMS are related to the properties of the ions to be tested. For example, high ion beam current and acceleration voltage are required to sputter the secondary ions from REEs. And under these conditions, other metal ions can also be detected and imaged.

Rare earth elements refer to scandium, yttrium, and 15 lanthanide elements. Specifically, one or more of scandium, yttrium, lanthanum, cerium, praseodymium, neodymium, promethium, samarium, europium, gadolinium, terbium, dysprosium, holmium, erbium, thulium, ytterbium, and lutetium are included.

In a preferred embodiment, the method for preparing the plant slice includes: cutting a plant tissue into a small piece and subjecting the same to fixation treatment, thereby obtaining a fixed sample; and subjecting the fixed sample to dehydration treatment, infiltration and embedding treatment, and slicing treatment successively, thereby gradually obtaining a dehydrated sample, an infiltrated and embedded sample, and the plant slice. More preferably, for leaf samples, pieces are cut along the direction parallel to the main leaf vein and the subsequent operations such as fixation treatment, etc., are carried out thereto to obtain the plant leaf samples suitable for the above methods.

In a preferred embodiment, the fixation treatment includes pre-fixation treatment and post-fixation treatment.

In a preferred embodiment, the pre-fixation treatment includes: pre-fixing the small piece using polyformaldehyde and glutaraldehyde to obtain a pre-fixed sample.

In a preferred embodiment, the post-fixation treatment includes: post-fixing the pre-fixed sample using an osmic acid solution to obtain a post-fixed sample.

In a preferred embodiment, the dehydration treatment includes: dehydrating the fixed sample using ethanol solution concentrations that with gradually increasing sequentially to obtain a dehydrated sample.

In a preferred embodiment, the infiltration and embedding treatment includes: using propylene oxide and an embedding agent for the infiltration and embedding of the dehydrated sample, resulting in an infiltrated and embedded sample.

In a preferred embodiment, the slicing treatment includes: slicing the infiltrated and embedded sample to obtain the sample slice.

In a preferred embodiment, the size of the small piece is 1-2 mm³.

In a preferred embodiment, the pre-fixation treatment includes: placing the small piece into a solution containing 1-4 wt % of paraformaldehyde and 0.5-5 wt % of glutaraldehyde, fixing same in a dark place at 15-25° C. for 2-3 hours or overnight at 4° C. to obtain the pre-fixed sample.

In a preferred embodiment, the post-fixation treatment includes: placing the pre-fixed sample in a 1-2 wt % osmic acid solution, fixing same in a dark place at 15-25° C. for 4 hours to obtain the post-fixed sample.

In a preferred embodiment, the dehydration treatment includes: subjecting the post-fixed sample to the dehydration treatment using the ethanol solutions of 30% (v/v), 50% (v/v), 70% (v/v), 90% (v/v), 100% (v/v), and 100% (v/v) (after treatment with a 90% (v/v) ethanol solution, the sample are further treated twice with 100% (v/v) ethanol solution), sequentially, wherein treatment times for the ethanol solutions ranging from 30% (v/v) to 90% (v/v) are 15 minutes each, and treatment times for the 100% (v/v) ethanol solution are 20 minutes each, thereby obtaining the dehydrated sample.

In a preferred embodiment, the infiltration and embedding treatment includes: sequentially treating the dehydrated sample with propylene oxide at 15-25° C. for 1 hour, with gradient mixed solutions of propylene oxide and an embedding agent at 15-25° C. for 4 hours, respectively, with an embedding agent at 15-25° C. for 8 hours, then polymerizing same at 60° C. for 48 hours to obtain the infiltrated and embedded sample.

The specific components of the embedding agent used in this application include: an aliphatic epoxy resin (3,4-epoxy cyclohexene methyl, ERL-4221), a hardener (nonyl succinic anhydride, NSA), an epoxy plasticizer (a polymer containing α-hydro-ω-hydroxypoly[oxy(methyl-1,2-ethylene)](chloromethyl)oxirane, DER-736), and a catalyst (N,N-dimethylethanolamine, DEMA); the mass ratio of ERL-4221 to NSA to DER-736 per 10 g of the embedding agent is 1:2:1, and the DEMA is 100 μL. Other embedding agents in the prior art can also be used or the components in the above-mentioned embedding agents can be flexibly adjusted, as long as the embedding effect can be achieved.

In a preferred embodiment, the gradient mixed solutions of propylene oxide and an embedding agent includes mixed solutions of propylene oxide and an embedding agent with a volume ratio of 2:1, 1:1, and 1:2, or 3:1, 1:1, and 1:3, sequentially. The sample is treated using the above mentioned mixed solutions with a concentration gradient, and the concentration of the embedding agent in the mixed solution continuously increases to ensure that the embedding agent completely penetrates into the sample tissues.

In a preferred embodiment, a thickness of the sample slice is 80-150 nm.

With the aforementioned method, slice sample suitable for electron microscopy imaging and ion beam bombardment can be obtained through fixation, dehydration, penetration embedding, and slicing treatments.

In a preferred embodiment, before preparing the sample slice, the plant is treated with the metal ion, including: spraying a solution containing the metal ion onto a leaf or leaves of the plant until the droplets begin to fall.

All of the metal ions in the cell samples can be analyzed using the aforementioned method, thereby achieving the research on the transport and accumulation mechanisms, etc., of the metal ions in cells, and it is not necessary to use metal ions for the pre-treatment of cells. Using the aforementioned treatment methods, spraying a solution containing the metal ions onto a leaf or leaves can achieve rapid entry of metal ions, especially rare earth metals, into cells, simulating the processes such as absorption and transport, etc., of metal ions by plants in the real growth state.

In a preferred embodiment, a concentration of the metal ion in the solution is 1 nmol/L to 1 mmol/L.

Using the aforementioned treatment methods, by spraying a solution containing specific metal ions onto the blank plant leaves to provide these metal ions to the cells of plant leaves, enabling plant cells to spontaneously absorb, transport, and accumulate the metal ions. This allows metal ions to be distributed in different subcellular structures of plant cells, facilitating subsequent research on the localization of metals in subcellular structures, as well as the absorption, transport, and accumulation mechanisms, etc., of metal elements by plant cells.

For other types of samples, by flexibly selecting the preparation methods in the prior art to prepare the samples for electron microscopy, samples or sample slices for the aforementioned subcellular self-tracer ion imaging and localization method of metal elements can also be obtained.

In a preferred embodiment, when the SEM is used for the subcellular structure imaging, an electron beam current is 21 pA to 1.4 nA, an acceleration voltage is 3-10 kV, a scanning speed is 1-100 μs, and a magnification factor is 2,500 to 5,000 folds.

In a preferred embodiment, when the FIB-TOF-SIMS is used for the surface bombardment, the secondary ion signal detection, and the ion imaging of the subcellular structure, an ion beam current is 24-80 pA and an acceleration voltage is 30 kV, and a number of images collected is 50-200.

By utilizing the above mentioned SEM parameters, it is possible to clearly develop the subcellular structure of plant cells and reduce the impact of electron beams emitted by SEM on cells and subcellular structures, providing a reference for accurate ion beam bombardment and localization of metal ions in subcellular structures subsequently. In the process of using FIB-TOF-SIMS, imaging images, pixels, and collection quantity can be flexibly selected as needed. The imaging image resolution selected in this application is 512×442, with pixel binning of 4, and a total of 50-200 images are collected.

By utilizing the parameters of FIB and TOF-SIMS mentioned above, precise surface bombardment of subcellular structures can be achieved, and secondary ion signals generated when bombarding the metal ions can be captured and detected, thereby accurately locating the occurrence position of metal ions on the subcellular structures and achieving ion imaging and localization at the subcellular level.

Although devices such as SEM, FIB, and TOF-SIMS, etc., are widely used for relevant ion imaging of solid material surfaces such as rock-soil, graphite, ceramics, and metals, etc., in the prior art. However, those skilled in this research field have not considered how to connect the above devices in series to obtain the localization and imaging of metal ions in biological cells, especially plant cells and subcells, and it is also difficult to predict what kind of assembling device can be obtained through a structure in series, and what kind of technical effects can be achieved therefrom. That is, in the aforementioned method, if the parameters of the SEM-FIB-TOF-SIMS system of the assembling device exceed the above preferred parameter range, or the steps used in the preparation method for sample slices (especially plant slices) exceed the above preferred range, the subcellular self-tracer ion imaging and localization effect of metal elements in plants is poor, and even the localization and imaging of ions in the subcellular structures cannot be achieved.

In a preferred embodiment, after the subcellular structure imaging and the ion imaging are performed, outputting an image area and a data sheet of the image area, and drawing the mass spectrometry image in the image area as described in the data table to represent a relative abundance of the metal ion.

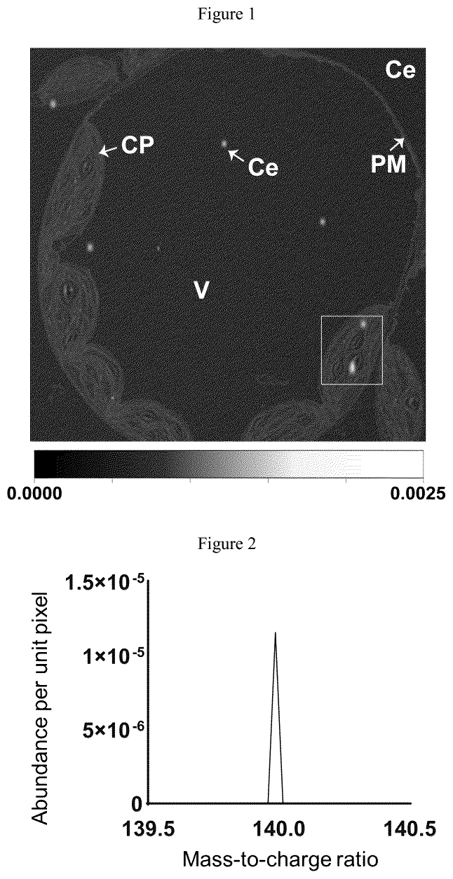

Preferably, the TOF-SIMS explorer software is used to capture the image area of interest and export a data sheet of all parameters of the image area of interest; and drawing software such as Origin2022, etc., are used to draw a mass spectrometry image of the relative abundance of the target metal ions in the image area of interest, wherein the peak height in the mass spectrometry image can trace the relative content or abundance of the ions to be detected in the target area. The SEM image and the ion image of FIB-TOF-SIM in the output images are overlaid using drawing software such as Adobe Photoshop, so that the image shown in FIG. 1 can be presented, achieving ion imaging and localization at the subcellular level.

The beneficial effects of this application will be further explained in detail below in conjunction with specific examples.

### Example 1

Specific steps of the subcellular self-tracer ion imaging and localization method for rare earth element cerium (Ce) in leaves of Arabidopsis thaliana were as follows:
(1) Treating plants with Ce: 80 μmol·L⁻¹ of cerium chloride (CeCl₃) was evenly sprayed onto the leaves of Arabidopsis thaliana until the droplets began to fall;
(2) Sampling: 2 mm³ of small pieces were cut along the direction parallel to the main leaf vein of the leaves of Arabidopsis thaliana;
(3) Pre-fixation: the sample was placed in a solution containing 4 wt % of paraformaldehyde (Shanghai Acmec Biochemical Technology Co., Ltd., P39200) and 5 wt % of glutaraldehyde, and fixed in a dark place overnight at 4° C.;
(4) Post-fixation: the sample was placed in a 1 wt % osmic acid solution and fixed in a dark place at 25° C. for 4 hours;
(5) Dehydration: the fixed samples were subjected to dehydration treatment using ethanol solutions with a volume ratio of 30%, 50%, 70%, 90%, 100%, and 100%, sequentially, wherein the treatment times for the ethanol solutions ranging from 30% to 90% were 15 minutes each, and the treatment times for the 100% ethanol solution are 20 minutes each;
(6) Infiltration and embedding: the dehydrated sample was sequentially treated with propylene oxide at 25° C. for 1 hour, with gradient mixed solutions of propylene oxide and an embedding agent (v:v=2:1,1:1,1:2) at 25° C. for 4 hours, respectively, with an embedding agent at 25° C. for 8 hours, and then polymerized at 60° C. for 48 hours; and the specific components of the embedding agent included: an aliphatic epoxy resin (3,4-epoxy cyclohexene methyl, ERL-4221), a hardener (nonyl succinic anhydride, NSA), an epoxy plasticizer (a polymer containing α-hydro-ω-hydroxypoly[oxy (methyl-1,2-ethylene)](chloromethyl)oxirane, DER-736), and a catalyst (N,N-dimethylethanolamine, DEMA); the mass ratio of ERL-4221 to NSA to DER-736 per 10 g of the embedding agent was 1:2:1, and the DEMA was 100 μL.
(7) Slicing: the embedded sample was cut into ultra-thin slices with a thickness of 120 nm;
(8) Subcellular structure imaging of a plant: SEM (Helios5CX, (Thermo Fisher Scientific, the United States of America)) was used to develop the subcellular structure of a plant, the electron beam current was 0.35 nA, the acceleration voltage was 5 kV, the scanning speed was 15 us, and the magnification factor was 3,500 folds;
(9) Ion imaging: FIB (Helios5CX, (Thermo Fisher Scientific, the United States of America) was used to bombard the surface of the sample, the ion beam current was 40 pA and the acceleration voltage was 30 kV; the secondary ions excited were detected by the time-of-flight secondary ion mass spectrometry (TOF-SIMS) to obtain the ion information in the analysis area and imaged, wherein the image resolution was 512×442, with pixel binning of 4, and a total of 100 images were collected.
(10) The TOF-SIMS explorer software was used to capture the image area of interest and a data sheet of all parameters of the image area of interest was exported.
(11) The drawing software Origin 2022 was used to draw a mass spectrometry image of the target metal ions in the image area of interest.

In the Example 1 of the present application, the subcellular self-tracer ion imaging of Ce in leaves of Arabidopsis thaliana was obtained, and the results of which are shown in FIG. 1. As shown in FIG. 1, after the leaves of Arabidopsis thaliana were treated with 80 μmol·L⁻¹ of CeCl₃, Ce was localized on the plasma membrane (PM), vacuole (V), and chloroplast (CP) of leaves of Arabidopsis thaliana. The mass spectrometry image of the relative abundance of Ce in the chloroplast (rectangular box area in FIG. 1) of leaves of Arabidopsis thaliana was also obtained, and the results of which are shown in FIG. 2.

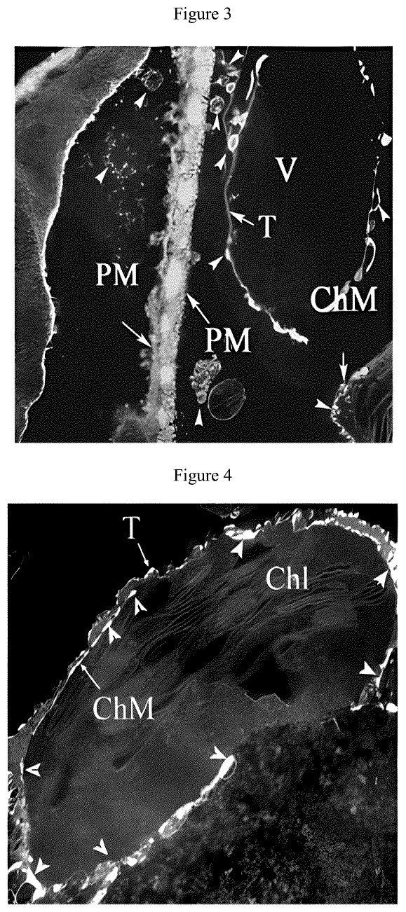

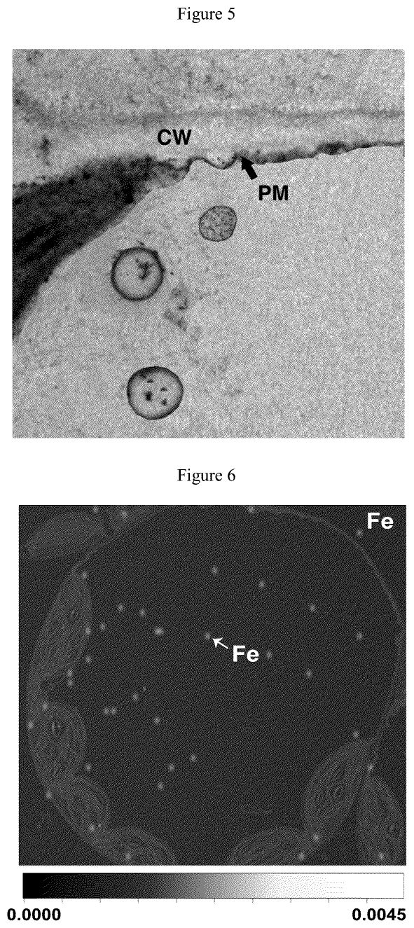

Combining with the results obtained from the rare earth radioactive isotope self-tracer imaging (FIGS. 3 and 4, from Wang L, Li J, Zhou Q, et al. Rare earth elements activate endocytosis in plant cells [J]. Proceedings of the National Academy of Sciences, 2014, 111(35): 12936-12941.) and the rare earth non-radioactive isotope self-tracer imaging (FIG. 5, from Cheng M, Wang L, Zhou Q, et al. Lanthanum(III) triggers AtrbohD- and jasmonic acid-dependent systemic endocytosis in plants [J]. Nature Communications, 2021, 12(1): 4327.) in the prior art, it can be known that the method of the present application can quickly obtain clear images containing the subcellular localization information of rare earth elements in Arabidopsis thaliana.

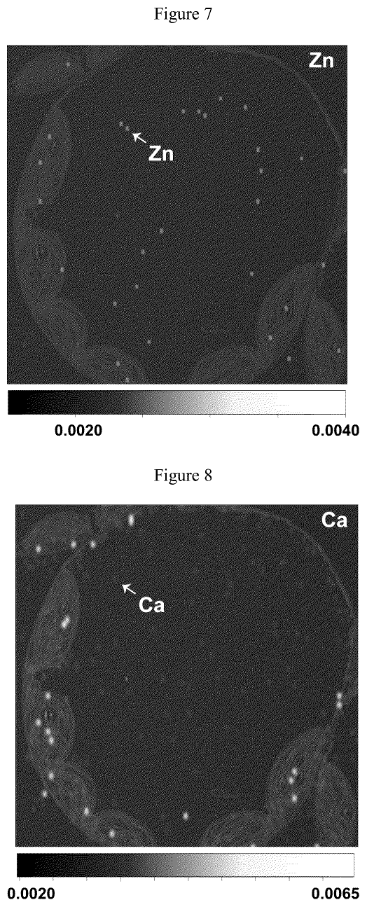

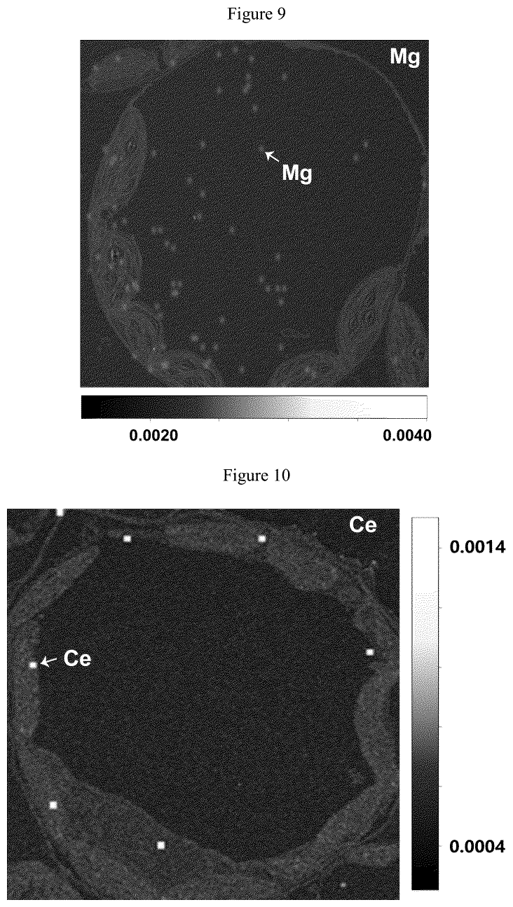

In Example 1 of the present application, the subcellular self-tracer ion imaging of other transition metal elements [iron (Fe), and zinc (Zn)] and non-transition metal elements [calcium (Ca), and magnesium (Mg)] in leaves of Arabidopsis thaliana was also obtained. The results are shown in FIGS. 6, 7, 8, and 9.

### Example 2

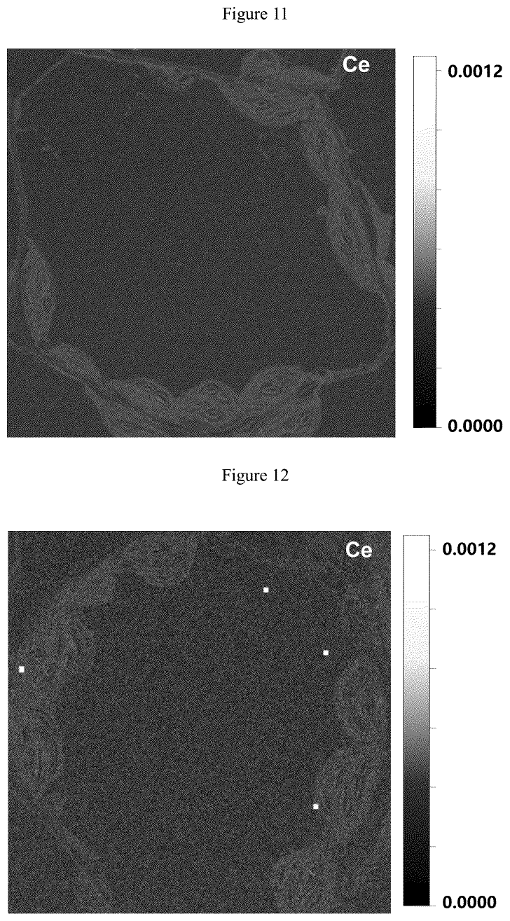

The subcellular self-tracer ion imaging and localization for Ce in leaves of corn (Zea mays). The steps in this Example were identical to those in Example 1, the effect was consistent with Example 1, and the results are shown in FIG. 10.

### Example 3

The subcellular self-tracer ion imaging and localization for Ce in leaves of Arabidopsis thaliana. In this Example, 0.01 nmol L⁻¹ of CeCl₃ was evenly sprayed onto the leaves of Arabidopsis thaliana. In addition, other steps were identical to those in Example 1, and its result was shown in FIG. 11. The results showed that compared to Example 1, the subcellular self-tracer ion imaging of Ce in leaves of Arabidopsis thaliana cannot be obtained.

### Example 4

The subcellular self-tracer ion imaging and localization for Ce in leaves of Arabidopsis thaliana. In this Example, the scanning speed was 500 ns when the SEM was used for imaging. In addition, other steps were identical to those in Example 1, and its result was shown in FIG. 12. The results showed that compared to Example 1, the clear subcellular self-tracer ion imaging of Ce in leaves of Arabidopsis thaliana cannot be obtained.

### Example 5

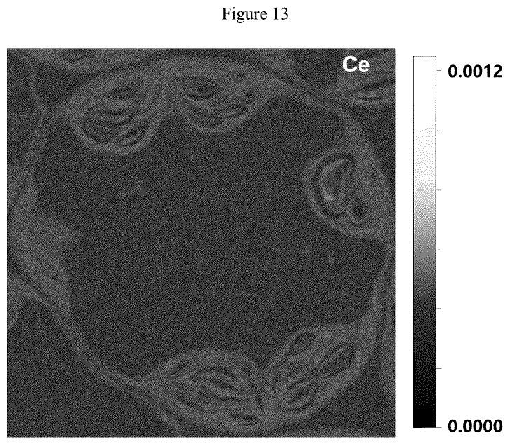

The subcellular self-tracer ion imaging and localization for Ce in leaves of Arabidopsis thaliana. In this Example, a total of 25 images were collected when FIB-TOF-SIMS was used for surface bombardment, secondary ion signal detection, and ion imaging of the sample. In addition, other steps were identical to those in Example 1, and its result was shown in FIG. 13. The results showed that compared to Example 1, the subcellular self-tracer ion imaging of Ce (III) in leaves of Arabidopsis thaliana cannot be obtained.

### Example 6

The subcellular self-tracer ion imaging and localization for Ce in leaves of Arabidopsis thaliana. In this Example, the acceleration voltage was 2 kV when the SEM was used for imaging. In addition, other steps were identical to those in Example 1. The results showed that compared to Example 1, the clear subcellular self-tracer ion imaging of Ce in leaves of Arabidopsis thaliana cannot be obtained.

### Example 7

The subcellular self-tracer ion imaging and localization for Ce in leaves of Arabidopsis thaliana. In this Example, the magnification factor was 1,000 folds when the SEM was used for imaging. In addition, other steps were identical to those in Example 1. The results showed that compared to Example 1, the clear subcellular self-tracer ion imaging of Ce in leaves of Arabidopsis thaliana cannot be obtained.

### Example 8

The subcellular self-tracer ion imaging and localization for Ce in leaves of Arabidopsis thaliana. In this Example, the ion beam current was 7.7 pA when FIB-TOF-SIMS was used for surface bombardment, secondary ion signal detection, and ion imaging of the sample. In addition, other steps were identical to those in Example 1. The results showed that compared to Example 1, the subcellular self-tracer ion imaging of Ce in leaves of Arabidopsis thaliana cannot be obtained.

From the above descriptions, it can be seen that the Examples of the present application have achieved the following technical effects: the subcellular self-tracer ion imaging and localization method of metal elements disclosed in this application has the advantages of simple and fast operation, clear images, good repeatability, and high safety, etc. It is suitable for cells from various biological sources and various metal ions, and can achieve imaging and localization of metal ions at the subcellular structure level. Especially, it not only can be applied for tracing and imaging the subcellular localization of transition metal ions with stable valence in various plants, but also can be applied for tracing and imaging the subcellular localization of non-transition metal ions with stable valence in various plants.

The above contents only describe the preferred examples of the present application, and are not intended to limit the present application. For those skilled in the art, various modifications and changes can be made to the present application. Any modifications, equivalent substitutions, improvements, and the like made within the spirit and principle of the application shall be included within the scope of protection of the application.

1. A subcellular self-tracer ion imaging and localization method of metal elements, wherein, the method comprises:
using an SEM-FIB-TOF-SIMS system to perform a subcellular structure imaging and a metal ion imaging on a sample slice,
wherein in the SEM-FIB-TOF-SIMS system, the scanning electron microscope (SEM) is used to perform subcellular structure imaging on the sample slice; and
the focused ion beam (FIB) is used to perform surface bombardment on the subcellular structure, and the secondary ions excited are detected by a time-of-flight secondary ion mass spectrometry (TOF-SIMS) to obtain an ion information in an analysis area and imaged.

2. The method according to claim 1, wherein the sample slice comprises a plant slice, an animal tissue slice, a bacterial slice or a fungal sample, and correspondingly, the subcellular structure comprises a plant subcellular structure, an animal subcellular structure, a bacterial subcellular structure, or a fungal subcellular structure.

3. The method according to claim 1, wherein the metal ion comprises a transition metal element ion or a non-transition metal element ion.

4. The method according to claim 3, wherein the transition metal element ion comprises a rare earth element ion.

5. The method according to claim 2, wherein the method for preparing the plant slice comprises:
cutting a plant tissue into a small piece and subjecting the same to fixation treatment, thereby obtaining a fixed sample; and
subjecting the fixed sample to dehydration treatment, infiltration and embedding treatment, and slicing treatment successively, thereby gradually obtaining a dehydrated sample, an infiltrated and embedded sample, and the plant slice.

6. The method according to claim 5, wherein the fixation treatment comprises pre-fixation treatment and post-fixation treatment.

7. The method according to claim 6, wherein the pre-fixation treatment comprises: pre-fixing the small piece using polyformaldehyde and glutaraldehyde to obtain a pre-fixed sample;
preferably, the post-fixation treatment comprises: post-fixing the pre-fixed sample using an osmic acid solution to obtain a post-fixed sample.

8. The method according to claim 5, wherein the dehydration treatment comprises: dehydrating the fixed sample using ethanol solutions with concentrations that gradually increasing sequentially to obtain the dehydrated sample.

9. The method according to claim 5, wherein the infiltration and embedding treatment comprises:
using propylene oxide and an embedding agent for the infiltration and embedding of the dehydrated sample, resulting in the infiltrated and embedded sample.

10. The method according to claim 5, wherein the slicing treatment comprises: slicing the infiltrated and embedded sample to obtain the plant slice.

11. The method according to claim 5, wherein before preparing the plant slice, the plant is treated with the metal ion, comprising: spraying a solution comprising the metal ion onto a leaf or leaves of the plant until droplets begin to fall.

12. The method according to claim 11, wherein a concentration of the metal ion in the solution is 1 nmol/L to 1 mmol/L.

13. The method according to claim 6, wherein a size of the small piece is 1-2 mm³.

14. The method according to claim 7, wherein the pre-fixation treatment comprises: placing the small piece into a solution comprising 1-4 wt % of paraformaldehyde and 0.5-5 wt % of glutaraldehyde, fixing same in a dark place at 15-25° C. for 2-3 hours or overnight at 4° C. to obtain the pre-fixed sample; and preferably, the post-fixation treatment comprises: placing the pre-fixed sample in a 1-2 wt % osmic acid solution, fixing same in a dark place at 15-25° C. for 4 hours to obtain the post-fixed sample.

15. The method according to claim 8, wherein the dehydration treatment comprises: subjecting the post-fixed sample to the dehydration treatment using the ethanol solutions of 30% (v/v), 50% (v/v), 70% (v/v), 90% (v/v), 100% (v/v), and 100% (v/v), sequentially, wherein treatment times for the ethanol solutions ranging from 30% (v/v) to 90% (v/v) are 15 minutes each, and treatment times for the 100% (v/v) ethanol solution are 20 minutes each thereby obtaining the dehydrated sample.

16. The method according to claim 9, wherein the infiltration and embedding treatment comprises:
sequentially treating the dehydrated sample with propylene oxide at 15-25° C. for 1 hour, with gradient mixed solutions of propylene oxide and an embedding agent at 15-25° C. for 4 hours, respectively, with an embedding agent at 15-25° C. for 8 hours, then polymerizing same at 60° C. for 48 hours to obtain the infiltrated and embedded sample.

17. The method according to claim 10, wherein a thickness of the plant slice is 80-150 nm.

18. The method according to claim 1 wherein, when the SEM is used for the subcellular structure imaging, an electron beam current is 21 pA to 1.4 nA, an acceleration voltage is 3-10 kV, a scanning speed is 1-100 μs, and a magnification factor is 2,500 to 5,000 folds.

19. The method according to claim 1, wherein, when the FIB-TOF-SIMS is used for the surface bombardment, the secondary ion signal detection, and the ion imaging of the subcellular structure, an ion beam current is 24-80 pA and an acceleration voltage is 30 kV, and a number of images collected is 50-200.

20. The method according to claim 1 wherein, after the subcellular structure imaging and the ion imaging are performed, outputting an image area and a data sheet of the image area, and drawing the mass spectrometry image in the image area as described in the data table to represent a relative abundance of the metal ion.
